(19) United States
(12) Patent Application Publication
(10) Pub. No.: US 2021/0386735 A1
(43) Pub. Date: Dec. 16, 2021

(54) Methods of Treating Acute HCV

(71) Applicants: AbbVie Inc., North Chicago, IL (US); NewSouth Innovations PTY Limited, Sydney (AU)

(72) Inventors: Gail Matthews, Kensington (AU); Gregory Dore, Kensington (AU); Mark Nelson, Chelsea (GB); Fernando Franciosi, Libertyville, IL (US); Ana Gabriela Pires Dos Santos, Round Lake, IL (US)

(21) Appl. No.: 17/295,181

(22) PCT Filed: Nov. 20, 2019
(86) PCT No.: PCT/US2019/062407

§ 371 Date: May 19, 2021

Related U.S. Application Data
(60) Provisional application No. 62/769,908, filed on Nov. 20, 2018.
(60) Provisional application No. 62/831,231, filed on Apr. 9, 2019.

(51) Int. Cl. A61K 31/4985; A61K 31/454; A61P 31/18

(52) U.S. Cl. CPC A61K 31/4985; A61P 31/18; A61K 31/454

(57) ABSTRACT

The present invention features interferon-free therapies for the treatment of acute HCV. Preferably, the treatment is over a shorter duration of treatment, such as 6 weeks. In one aspect, the treatment comprises administering two direct acting antiviral agents to a subject with acute HCV infection, wherein the treatment lasts for 6 weeks and does not include administration of either interferon or ribavirin, and said direct acting antiviral agents comprise (a) glecaprevir or a pharmaceutically acceptable salt thereof and (b) pibrentasvir or a pharmaceutically acceptable salt thereof.

### JOINT RESEARCH AGREEMENT

Subject matter disclosed in this application was made by or on behalf of AbbVie Inc. and/or The Kirby Institute, Sydney, Australia, whom are parties to a joint research agreement that was in effect on or before the effective filing date of this application, and such subject matter was made as a result of activities undertaken within the scope of the joint research agreement.

### FIELD OF THE INVENTION

The present invention relates to interferon-free and ribavirin-free treatment for acute infection of hepatitis C virus (HCV).

### BACKGROUND OF THE INVENTION

HCV is an RNA virus belonging to the Hepacivirus genus in the Flaviviridae family. The enveloped HCV virion contains a positive stranded RNA genome encoding all known virus-specific proteins in a single, uninterrupted, open reading frame. The open reading frame comprises approximately 9500 nucleotides and encodes a single large polyprotein of about 3000 amino acids. The polyprotein comprises a core protein, envelope proteins E1 and E2, a membrane bound protein p7, and the non-structural proteins NS2, NS3, NS4A, NS4B, NS5A and NS5B.

Chronic HCV infection is associated with progressive liver pathology, including cirrhosis and hepatocellular carcinoma. In the past, chronic hepatitis C was treated with peginterferon-alpha in combination with ribavirin, which had substantial limitations of efficacy and tolerability. Recently, Glecaprevir-pibrentasvir was approved for treatment of chronic HCV infection in treatment-naïve individuals without cirrhosis for a duration lasting only eight weeks. Further, all other currently approved treatments, sofosbuvir, ledipasvir, velpatasvir, voxilaprevir, daclatasvir, elbasvir, grazoprevir, simeprevir are indicated for the treatment of chronic HCV. While numerous drugs are indicated for treatment of chronic HCV, no drug thus far is indicated for acute treatment of HCV. Moreover, since the goal of World Health Organization is to eliminate HCV as a public threat by 2030, therefore, there is a need for new therapies and treatment options to treat acute HCV infection, wherein duration is further shortened for compliance, without negative impact on efficacy, viral breakthrough, resistance, recurrent viraemia, virological relapse or reinfection.

### BRIEF SUMMARY OF THE INVENTION

The present invention relates to interferon-free and ribavirin-free treatment for acute infection of hepatitis C virus (HCV). In one embodiment, the present invention provides a method for treatment for acute HCV, comprising administering two direct acting antiviral agents (DAAs) to a HCV patient, wherein said treatment does not include administration of either interferon or ribavirin to said patient, and said treatment lasts for 6 weeks, and wherein said two DAAs comprise (1) glecaprevir or a pharmaceutically acceptable salt thereof and (2) pibrentasvir or a pharmaceutically acceptable salt thereof. In another embodiment of the invention, the method provides that said patient has other co-morbid conditions, such as said patient is HCV-HIV co-infected. In another embodiment of the invention, the method provides that said patient is without cirrhosis. In another embodiment of the invention, the method provides that said patient is with compensated cirrhosis. In another embodiment of the invention, the method provides that said patient is a treatment-naïve patient. In another embodiment of the invention, the method provides that said patient is an interferon non-responder. In another embodiment of the invention, the method provides that said patient is a kidney or liver transplant patient. In another embodiment of the invention, the method provides that said patient has any degree of renal impairment.

Another embodiment of the invention provides a method of treatment of acute HCV with glecaprevir and pibrentasvir for a duration of 6 weeks wherein said patient is infected with HCV genotype 1. In another embodiment, said patient is infected with HCV genotype 1a. In yet another embodiment, said patient is infected with HCV genotype 2. In yet another embodiment, said patient is infected with HCV genotype 3. In yet another embodiment, said patient is infected with HCV genotype 4. In yet another embodiment, said patient is infected with HCV genotype 5. In yet another embodiment, said patient is infected with HCV genotype 6. In all the above embodiments, wherein the patients have HCV of genotypes including 1, 2, 3, 4, 5 or 6 and their subgenotypes, said patient may further have other co-morbidities, such as HCV-HIV co-infection, or, liver without cirrhosis or with compensated cirrhosis, or, renal or kidney transplants, or, any degree of renal impairment, or permutations and combinations thereof. Further said patient may be treatment-naïve patient or an interferon non-responder, or permutations and combinations thereof. For example, said patient may have acute HCV infection with genotype 1, and may further have renal impairment, or said patient may have acute HCV infection of genotype 4, and may further have HIV co-infection and/or may be an interferon non-responder.

In some embodiments, the invention provides at least two direct acting antiviral agents (DAAs) for use in a method of treating acute HCV in a patient, wherein said two DAAs comprise (1) glecaprevir or a pharmaceutically acceptable salt thereof and (2) pibrentasvir or a pharmaceutically acceptable salt thereof and said treatment lasts for a duration of 6 weeks. Acute HCV, as opposed to recent or chronic HCV, refers to HCV infection having a duration of less than 6 months. In other words, acute hepatitis C virus (HCV) infection is defined as the 6 month time period following acquisition of hepatitis C virus. The patient may exhibit symptoms in this period, or may be asymptomatic.

The estimated date of clinical infection is calculated as 6 weeks prior to seroconversion illness or ALT>10×ULN. The estimated date of asymptomatic infection is calculated as the midpoint between the last negative anti-HCV Ab or HCV RNA and the 1st positive anti-HCV Ab or HCV RNA. For participants who were anti-HCV Ab negative and HCV RNA positive at screening, the estimated date of infection was 6 weeks prior to enrolment. The primary efficacy endpoint was SVR12, defined as HCV RNA below the lower limit of quantitation (LLoQ; target not detected or target detected, not quantifiable) at post-treatment week 12.

Accordingly, in some embodiments, the invention provides at least two direct acting antiviral agents (DAAs) for use in a method of treating acute HCV in a patient, wherein said two DAAs comprise (1) glecaprevir or a pharmaceutically acceptable salt thereof and (2) pibrentasvir or a pharmaceutically acceptable salt thereof; wherein said method includes determining the date of seroconversion of said patient and beginning treatment within 4.5 months of said date of seroconversion; wherein the treatment comprises administering effective amounts of said at least two DAAs for a duration of 6 weeks.

In other embodiments, the invention provides at least two direct acting antiviral agents (DAAs) for use in a method of treating acute HCV in a patient, said patient having a clinically determined date of HCV seroconversion in the previous 4.5 months, wherein said two DAAs comprise (1) glecaprevir or a pharmaceutically acceptable salt thereof and (2) pibrentasvir or a pharmaceutically acceptable salt thereof, and wherein said treatment lasts for a duration of 6 weeks.

Acute HCV infection may also be defined as within six month of ALT>10×ULN. Methods of determining ALT and ULN are known in the art. Accordingly, in some embodiments, the invention provides at least two direct acting antiviral agents (DAAs) for use in a method of treating acute HCV in a patient, wherein said two DAAs comprise (1) glecaprevir or a pharmaceutically acceptable salt thereof and (2) pibrentasvir or a pharmaceutically acceptable salt thereof; wherein said method includes determining the date of ALT>10×ULN of said patient and beginning treatment within 4.5 months of said date of ALT>10×ULN; wherein the treatment comprises administering effective amounts of said at least two DAAs for a duration of 6 weeks.

In other embodiments, the invention provides at least two direct acting antiviral agents (DAAs) for use in a method of treating acute HCV in a patient, said patient having a clinically determined date of ALT>10×ULN in the previous 4.5 months, wherein said two DAAs comprise (1) glecaprevir or a pharmaceutically acceptable salt thereof and (2) pibrentasvir or a pharmaceutically acceptable salt thereof, and wherein said treatment lasts for a duration of 6 weeks.

In the above-described embodiments, the patient may have other co-morbid conditions, such as said patient being HCV-HIV co-infected. The patient may have HCV of genotypes including 1, 2, 3, 4, 5 or 6 and their subgenotypes, and may be without cirrhosis or with compensated cirrhosis, or, renal or kidney transplants, or, any degree of renal impairment, or permutations and combinations thereof. Further said patient may be treatment-naïve patient or an interferon non-responder, or permutations and combinations thereof. For example, said patient may have acute HCV infection with genotype 1, and may further have renal impairment, or said patient may have acute HCV infection of genotype 4, and may further have HIV co-infection and/or may be an interferon non-responder.

In further embodiments, the invention relates to treatment of recent HCV infection. Recent HCV refers to duration of infection of less than 12 months, for example it may refer to a duration of infection of 6 months or more and less than 12 months. Recent HCV infection may refer to recent primary HCV infection or recent HCV reinfection. Accordingly, the invention may provide at least two direct acting antiviral agents (DAAs) for use in a method of treating recent HCV infection HCV in a patient, wherein said two DAAs comprise (1) glecaprevir or a pharmaceutically acceptable salt thereof and (2) pibrentasvir or a pharmaceutically acceptable salt thereof and said treatment lasts for a duration of 6 weeks. Recent HCV infection may be a recent primary HCV infection or a recent HCV reinfection.

In some embodiments, the recent HCV infection is recent primary HCV infection. A patient may be categorized as having recent primary HCV infection if the 1^{st} positive anti-HCV Ab and/or HCV RNA determination was made within the 6 months preceding the start of treatment according to a method described herein; and if one or more of the following conditions is met: (i) HCV seroconversion within 18 months (ii) Acute clinical hepatitis within 12 months (jaundice or ALT>10×ULN) (iii) Acute asymptomatic hepatitis within 12 months (ALT>5×ULN). In some embodiments, the patient treated for recent HCV infection is categorized according to the preceding criteria.

In some embodiments, the recent HCV infection is recent reinfection. A patient may be categorized as having recent reinfection if a new positive HCV RNA determination was made the within the 6 months preceding the start of treatment according to a method described herein, following previous clearance. In other words positive anti-HCV Ab and undetectable HCV RNA on ≥2 occasions 6 months apart. In some embodiments, the patient treated for recent HCV infection is categorized according to the preceding criteria.

### DETAILED DESCRIPTION OF THE INVENTION

Glecaprevir-pibrentasvir has been approved for eight weeks for treatment of chronic HCV infection in treatment-naïve individuals without cirrhosis. This is the shortest duration available for any chronic HCV treatments. The object of this invention includes, amongst others to assess the efficacy of glecaprevir-pibrentasvir for six weeks in people with acute or recent HCV infection. Use of glecaprevir and pibrentasvir for the treatment of HCV is described in US patent applications, US20140275099 filed on Mar. 14, 2014, US20180177779, filed on Jul. 7, 2016; US20160317602, filed on Apr. 1, 2015; US20170151238 filed on Feb. 14, 2017, US20170360783 filed on Sep. 1, 2017 and US20170333428, filed on Aug. 2, 2017, all of which are incorporated herein by reference.

Methods: An open-label single-arm study was conducted in Australia, New Zealand and England, for adults with recent HCV (estimated duration of infection<12 months). Such subjects received glecaprevir-pibrentasvir 300 mg/120 mg daily for six weeks. Primary infection was defined by first positive anti-HCV antibody (Ab) and/or detectable HCV RNA within 6 months of enrolment and either acute clinical hepatitis within the past 12 months (symptomatic seroconversion illness or ALT (alanine aminotransferase) greater than 10 times the upper limit of normal [ULN]) or documented anti-HCV Ab seroconversion within 18 months. Reinfection was defined as new detectable HCV RNA within 6 months of enrolment and evidence of prior clearance (positive anti-HCV Ab and undetectable HCV RNA on ≥2 occasions). The primary endpoint was sustained virologic response at 12 weeks post treatment (SVR12) with efficacy endpoints reported in the intention-to treat (ITT) and per-protocol (PP) populations.

Results: Thirty people (median age 43 years, male 100%) received treatment, of whom 77% (n=23) were HIV-positive, 33% (n=10) had never injected drugs and 13% (n=4) had HCV reinfection. The majority were infected with HCV genotype 1 (80%, n=24), followed by genotype 4 (10%, n=3), 3 (7%, n=2) and 2 (3%, n=1). Median maximum ALT in the preceding 12 months was 381 U/L (range 26, 3087). Acute clinical hepatitis with ALT>10×ULN was reported in 73%; five (17%) had jaundice. At end of treatment, HCV RNA was below the limit of quantitation in 97% (n=29); one participant had quantifiable HCV RNA (21 IU/mL) and achieved SVR12. Of those who have reached post treatment week four (n=28), SVR4 ITT and PP were 93% and 100%, respectively (loss to follow up, n=2). Of those who have reached post treatment week 12 (n=22), SVR12 ITT and PP were 77% and 94%, respectively (detectable HCV RNA, n=1; death after SVR4, n=1; loss to follow up, n=3); sequencing was awaited in case of recurrent viraemia to determine if this represented relapse or reinfection (HCV RNA negative at SVR4).

Conclusion: Shortened duration pan-genotypic therapy with glecaprevir-pibrentasvir for six weeks was highly effective among HIV-positive and HIV-negative individuals with acute and recent HCV infection.

When a subject is diagnosed with acute HCV, then said subject may be treated with a shortened 6 weeks of treatment regimen of glecaprevir and pibrentasvir. All main genotypes of HCV, including sub-genotypes of 1, 2, 3, 4, 5 or 6 may be treatable, based on above data. Moreover, subjects that were HIV-HCV co-infected may also be treated with a shortened duration of 6 weeks of glecaprevir and pibrentasvir. This shortened treatment duration of 6 weeks for acute HCV may be especially useful where said patients have any degree of renal impairment, mild, moderate or severe. The shortened duration of treatment for acute HCV may also be used for subjects with compensated cirrhosis, including Child-Pugh A, Child Pugh B and others. This shortened treatment duration of 6 weeks may be useful for treating acute HCV in subjects with liver or kidney transplant.

In one embodiment, the present invention relates to interferon-free and ribavirin-free treatment for acute infection of hepatitis C virus (HCV). In one embodiment, the present invention provides a method for treatment for acute HCV, comprising administering two direct acting antiviral agents (DAAs) to a HCV patient, wherein said treatment does not include administration of either interferon or ribavirin to said patient, and said treatment lasts for 6 weeks, and wherein said two DAAs comprise (1) glecaprevir or a pharmaceutically acceptable salt thereof and (2) pibrentasvir or a pharmaceutically acceptable salt thereof. In another embodiment of the invention, the method provides that said patient has other co-morbid conditions, such as said patient is HCV-HIV co-infected. In another embodiment of the invention, the method provides that said patient is without cirrhosis. In another embodiment of the invention, the method provides that said patient is with compensated cirrhosis. In another embodiment of the invention, the method provides that said patient is a treatment-naïve patient. In another embodiment of the invention, the method provides that said patient is an interferon non-responder. In another embodiment of the invention, the method provides that said patient is a kidney or liver transplant patient. In another embodiment of the invention, the method provides that said patient has any degree of renal impairment.

Another embodiment of the invention provides a method of treatment of acute HCV with glecaprevir and pibrentasvir for a duration of 6 weeks wherein said patient is infected with HCV genotype 1. In another embodiment, said patient is infected with HCV genotype 1a. In yet another embodiment, said patient is infected with HCV genotype 2. In yet another embodiment, said patient is infected with HCV genotype 3. In yet another embodiment, said patient is infected with HCV genotype 4. In yet another embodiment, said patient is infected with HCV genotype 5. In yet another embodiment, said patient is infected with HCV genotype 6. In all the above embodiments, wherein the patients have HCV of genotypes including 1, 2, 3, 4, 5 or 6 and their subgenotypes, said patient may further have other co-morbidities, such as HCV-HIV co-infection, or, liver without cirrhosis or with compensated cirrhosis, or, renal or kidney transplants, or, any degree of renal impairment, or permutations and combinations thereof. Further said patient may be treatment-naïve patient or an interferon non-responder, or permutations and combinations thereof. For example, said patient may have acute HCV infection with genotype 1, and may further have renal impairment, or said patient may have acute HCV infection of genotype 4, and may further have HIV co-infection and/or may be an interferon non-responder.

The foregoing description of the present invention provides illustration and description, but is not intended to be exhaustive or to limit the invention to the precise one disclosed. Modifications and variations are possible in light of the above teachings or may be acquired from practice of the invention. Thus, it is noted that the scope of the invention is defined by the claims and their equivalents.

What is claimed is:

1. A method for treatment for acute Hepatitis C Virus (HCV), comprising administering two direct acting antiviral agents (DAAs) to said HCV patient, wherein said treatment does not include administration of either interferon or ribavirin to said patient, and said treatment lasts for 6 weeks, and wherein said two DAAs comprise (1) glecaprevir or a pharmaceutically acceptable salt thereof and (2) pibrentasvir or a pharmaceutically acceptable salt thereof.

2. The method according to claim 1, wherein said patient is HCV-HIV co-infected.

3. The method according to one of claims 1-2, wherein said patient is without cirrhosis.

4. The method according to one of claims 1-2, wherein said patient is with compensated cirrhosis.

5. The method according to one of claims 1-4, wherein said patient is a treatment-naïve patient.

6. The method according to one of claims 1-5, wherein said patient is an interferon non-responder.

7. The method according to one of claims 1-6, wherein said patient is kidney or liver transplant patient.

8. The method according to one of claims 1-7, wherein said patient any degree of renal impairment.

9. The method according to one of claims 1-8, wherein said patient is infected with HCV genotype 1.

10. The method according to one of claims 1-8, wherein said patient is infected with HCV genotype 1a.

11. The method according to one of claims 1-8, wherein said patient is infected with HCV genotype 2.

12. The method according to one of claims 1-8, wherein said patient is infected with HCV genotype 3.

13. The method according to one of claims 1-8, wherein said patient is infected with HCV genotype 4.

14. The method according to one of claims 1-8, wherein said patient is infected with HCV genotype 5.

15. The method according to one of claims 1-8, wherein said patient is infected with HCV genotype 6.
